(19) United States
(12) Patent Application Publication
(10) Pub. No.: US 2026/0235559 A1
(43) Pub. Date: Aug. 13, 2026

(54) ANALYSIS SYSTEM FOR HYDROCARBON COMPONENTS IN ULTRAPURE AMMONIA

(71) Applicant: Lansis Instrument (Shanghai) Co., Ltd, Shanghai (CN)

(72) Inventors: Chaoqing LI, Shanghai (CN); Fude WANG, Shanghai (CN); Jianhao LI, Shanghai (CN)

(73) Assignee: Lansis Instrument (Shanghai) Co., Ltd, Shanghai (CN)

(21) Appl. No.: 19/505,248

(22) PCT Filed: Dec. 1, 2023
(86) PCT No.: PCT/CN2023/135871

§ 371 Date: Jan. 23, 2026

(30) Foreign Application Priority Data
Oct. 12, 2023 (CN) 202311323506.1

(51) Int. Cl. G01N 30/08; G01N 30/20; G01N 30/68

(52) U.S. Cl. CPC G01N 30/08; G01N 30/20; G01N 30/68; G01N 2030/201

(57) ABSTRACT

An analysis system for hydrocarbon components in ultrapure ammonia. The analysis system comprises: a sample storage tank (1), an electronic refrigeration and concentration apparatus (7), a carrier gas apparatus (10) and a detection apparatus, wherein the electronic refrigeration and concentration apparatus (7) is connected to the sample storage tank (1), and the electronic refrigeration and concentration apparatus (7) is connected to the sample storage tank (1) is also connected to both the carrier gas apparatus (10) and the detection apparatus; and the detection apparatus comprises a chromatographic column (12) and a hydrogen flame ionization detector (13). The electronic refrigeration and concentration apparatus (7) refrigerates and concentrates samples, and on this basis, hydrocarbon components in ultrapure ammonia can be detected by means of the chromatographic column (12) and/or hydrogen flame ionization detector (13), without the need for using a pulse discharge helium ionization detector, thereby reducing the costs of detecting hydrocarbon components in ultrapure ammonia.

### CROSS-REFERENCE TO RELATED APPLICATION

This patent application is a national stage application of International Patent Application No. PCT/CN 2023/135871, filed on Dec. 1, 2023, which claims the priority of Chinese Patent Application No. 202311323506.1 filed with the China National Intellectual Property Administration on Oct. 12, 2023, and entitled “Analysis system for hydrocarbon components in ultrapure ammonia”, the entire content of which is incorporated by reference in the present application.

### TECHNICAL FIELD

The present disclosure relates to the technical field of component detection, and in particular to an analysis system for hydrocarbon components in ultrapure ammonia.

### BACKGROUND

In GB/T 14601-2009 Gas for electronic industry-Ammonia, it is required that in electronic grade products, the purity volume fraction of NH₃ is greater than or equal to 99.9995% and the content volume fraction of hydrocarbons (C₁-C₃) is less than 1 μ mol/mol, and in optoelectronic grade products, the purity volume fraction of NH₃ is greater than or equal to 99.99994% and the content volume fraction of hydrocarbons (C₁-C₃) is less than 0.05 μ mol/mol. Therefore, the detection of hydrocarbon content during the use of gas for electronic industry is crucial. At present, the detection of hydrogen components in ultrapure ammonia is generally conducted using a pulse discharge helium ionization detector, leading to high cost.

### SUMMARY

An objective of the present disclosure is to provide an analysis system for hydrocarbon components in ultrapure ammonia, so as to reduce the detection cost of hydrocarbon components in ultrapure ammonia.

To achieve the objective above, the present disclosure employs the following technical solution:

An analysis system for hydrocarbon components in ultrapure ammonia is provided in the present disclosure. The analysis system includes a sample storage tank, an electronic refrigeration and concentration apparatus, a carrier gas apparatus, and a detection apparatus.

The electronic refrigeration and concentration apparatus is connected to the sample storage tank, and is also connected to the carrier gas apparatus and the detection apparatus, respectively.

The electronic refrigeration concentration apparatus is used to refrigerate and concentrate a sample to be detected taken out from the sample storage tank.

The carrier gas apparatus is used to carry the sample to be detected after refrigeration and condensation in the electronic refrigeration and concentration apparatus into the detection apparatus.

The detection apparatus is used to detect hydrocarbon components in the sample to be detected after refrigeration and concentration.

The detection apparatus includes a chromatographic column, and a hydrogen flame ionization detector.

Alternatively, the analysis system further includes an automatic switching six-way valve.

The automatic switching six-way valve is connected between the electronic refrigeration and concentration apparatus and the sample storage tank, between the electronic refrigeration and concentration apparatus and the carrier gas apparatus, and between the electronic refrigeration and concentration apparatus and the detection apparatus, specifically as follows:

the sample storage tank is connected to a first port of the automatic switching six-way valve;
the electronic refrigeration and concentration apparatus is connected to a sixth port and a third port of the automatic switching six-way valve, respectively;
the carrier gas apparatus is connected to a fifth port of the automatic switching six-way valve; and
the detection apparatus is connected to a fourth port of the automatic switching six-way valve.

In the process of sample collection, the automatic switching six-way valve is in a first state, the first state is that the first port of the automatic switching six-way valve communicates with the sixth port, the second port communicates with the third port, and the fourth port communicates with the fifth port.

In the process of analysis and detection, the automatic switching six-way valve is in a second state, the second state is that the first port of the automatic switching six-way valve communicates with the second port, the third port communicates with the fourth port, and the fifth port communicates with the sixth port.

Alternatively, the analysis system further includes a sample recovery apparatus.

The sample recovery apparatus is connected to the second port of the automatic switching six-way valve.

Alternatively, the analysis system further includes a first three-way selector valve, a second three-way selector valve, and a third three-way selector valve.

The sample storage tank and the sample recovery apparatus are connected to the automatic switching six-way valve by means of the first three-way selector valve, the second three-way selector valve, and the third three-way selector valve, specifically as follows:
an interface a, an interface b and an interface c of the first three-way selector valve are connected to an interface a of the third three-way selector valve, the sample storage tank and the carrier gas apparatus, respectively;
an interface b and an interface c of the third three-way selector valve are connected to the first port of the automatic switching six-way valve and an interface b of the second three-way selector valve, respectively; and
an interface a and an interface c of the second three-way selector valve are connected to the sample recovery apparatus and the second port of the automatic switching six-way valve, respectively.

Alternatively, the analysis system further includes: a planar tee.

The carrier gas apparatus is connected to the automatic switching six-way valve by means of the planar tee, specifically as follows:
three interfaces of the planar tee are connected to the carrier gas apparatus, the interface c of the first three-way selector valve and the fifth port of the automatic switching six-way valve, respectively.

Alternatively, an outlet of the sample storage tank is provided with a first pressure relief valve.

Alternatively, an outlet of the carrier gas apparatus is provided with a second pressure relief valve.

According to specific embodiments of the present disclosure, the present disclosure has the following technical effects:

An analysis system for hydrocarbon components in ultrapure ammonia is provided by the embodiments of the present disclosure, including a sample storage tank, an electronic refrigeration and concentration apparatus, a carrier gas apparatus, and a detection apparatus. The electronic refrigeration and concentration apparatus is connected to the sample storage tank, and is also connected to the carrier gas apparatus and the detection apparatus, respectively. The electronic refrigeration concentration apparatus is used to refrigerate and concentrate a sample to be detected taken out from the sample storage tank. The carrier gas apparatus is used to carry the sample to be detected after refrigeration and condensation in the electronic refrigeration and concentration apparatus into the detection apparatus. The detection apparatus is used to detect hydrocarbon components in the sample to be detected after refrigeration and concentration. The detection apparatus includes a chromatographic column, and a hydrogen flame ionization detector. According to the embodiments of the present disclosure, the electronic refrigeration and concentration apparatus is used to refrigerate and concentrate a sample, and on the basis, the detection of hydrocarbon components in the ultrapure ammonia can be achieved by means of the chromatographic column and/or hydrogen flame ionization detector without using a pulse discharge helium ionization detector, and thus the detection cost of the hydrocarbon components in the ultrapure ammonia is reduced.

### BRIEF DESCRIPTION OF THE DRAWINGS

To describe the technical solutions in the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a state schematic diagram of an analysis system for hydrocarbon components in ultrapure ammonia in the process of sample collection according to embodiments of the present disclosure;

FIG. 2 is a state schematic diagram of an analysis system for hydrocarbon components in ultrapure ammonia in the process of analysis and detection according to embodiments of the present disclosure.

In the drawings:

1—sample storage tank; 2—first pressure relief valve; 3—first three-way selector valve; 4—second three-way selector valve; 5—third three-way selector valve; 6—sample recovery apparatus; 7—electronic refrigeration and concentration apparatus; 8—second pressure relief valve; 9—planar tee; 10—carrier gas apparatus; 11—automatic switching six-way valve; 12—chromatographic column; 13—hydrogen flame ionization detector.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

An objective of the present disclosure is to provide an analysis system for hydrocarbon components in ultrapure ammonia, so as to reduce the detection cost of the hydrocarbon components in ultrapure ammonia.

In order to make the objectives, technical solutions and advantages of the present disclosure more clearly, the present disclosure is further described in detail below with reference to the embodiments.

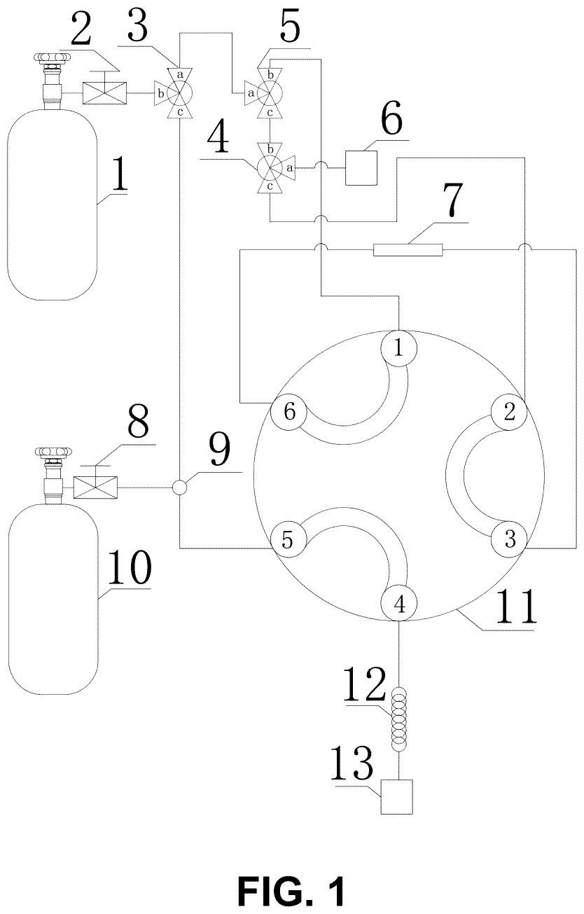

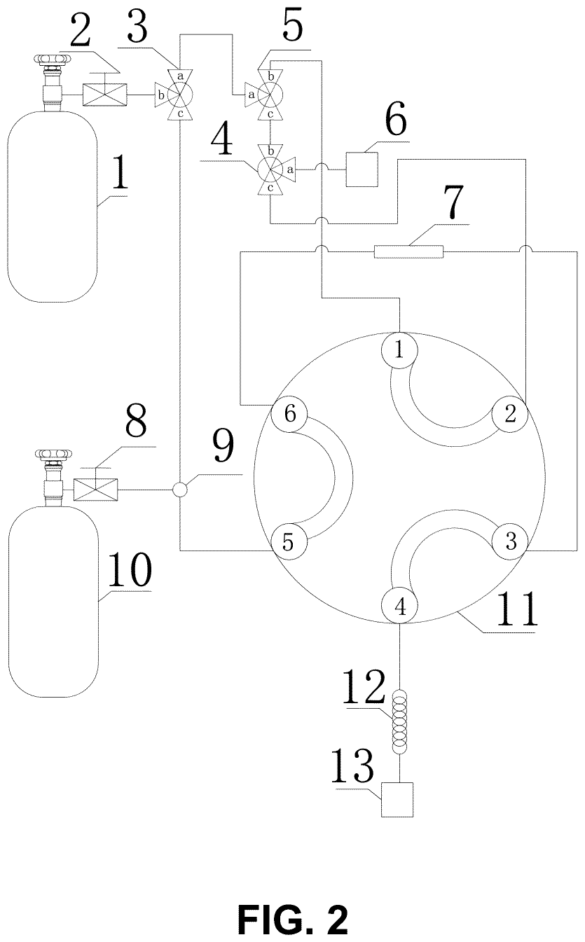

An analysis system for hydrocarbon components in ultrapure ammonia is provided by the embodiments of the present disclosure, as shown in FIG. 1 and FIG. 2, including a sample storage tank 1, an electronic refrigeration and concentration apparatus 7, a carrier gas apparatus 10, and a detection apparatus. The electronic refrigeration and concentration apparatus 7 is connected to the sample storage tank 1, and is also connected to the carrier gas apparatus 10 and the detection apparatus, respectively. The electronic refrigeration concentration apparatus 7 is used to refrigerate and concentrate a sample to be detected taken out from the sample storage tank 1. The carrier gas apparatus 10 is used to carry the sample to be detected after refrigeration and condensation in the electronic refrigeration and concentration apparatus 7 into the detection apparatus. The detection apparatus is used to detect hydrocarbon components in the sample to be detected after refrigeration and concentration. In the embodiments of the present disclosure, the detection apparatus includes a chromatographic column 12, and a hydrogen flame ionization detector 13.

In the embodiments of the present disclosure, the chromatographic column 12 is filled with porapak QS chromatographic support or other equivalent chromatographic supports.

The analysis system further includes: an automatic switching six-way valve 11. The automatic switching six-way valve 11 is connected between the electronic refrigeration and concentration apparatus 7 and the sample storage tank 1, between the electronic refrigeration and concentration apparatus 7 and the carrier gas apparatus 10, and between the electronic refrigeration and concentration apparatus 7 and the detection apparatus. Specifically, the sample storage tank 1 is connected a first port of the automatic switching six-way valve 11; the electronic refrigeration and concentration apparatus 7 is connected to a sixth port and a third port of the automatic switching six-way valve 11, respectively; the carrier gas apparatus 10 is connected to a fifth port of the automatic switching six-way valve 11; and the detection apparatus is connected to a fourth port of the automatic switching six-way valve 11. In the process of sample collection, the automatic switching six-way valve 11 is in a first state, the first state is that the first port of the automatic switching six-way valve 11 communicates with the sixth port, the second port communicates with the third port, and the fourth port communicates with the fifth port. In the process of analysis and detection, the automatic switching six-way valve 11 is in a second state, the second state is that the first port of the automatic switching six-way valve 11 communicates with the second port, the third port communicates with the fourth port, and the fifth port communicates with the sixth port. In the embodiments of the present disclosure, the first port, the second port, the third port, the fourth port, the fifth port and the sixth port correspond to the port \textcircled{1}, port \textcircled{2}, port \textcircled{3}, port \textcircled{4}, port \textcircled{5} and port \textcircled{6} in FIG. 1.

Exemplary, as a preferred embodiment, the analysis system further includes a sample recovery apparatus 6. The sample recovery apparatus 6 is connected to the second port of the automatic switching six-way valve 11. The analysis system further includes: a first three-way selector valve 3, a second three-way selector valve 4, and a third three-way selector valve 5. The sample storage tank 1 and the sample recovery apparatus 6 are connected to the automatic switching six-way valve 11 by means of the first three-way selector valve 3, the second three-way selector valve 4, and the third three-way selector valve 5. Specifically, an interface a, an interface b and an interface c of the first three-way selector valve 3 are connected to an interface a of the third three-way selector valve 5, the sample storage tank 1 and the carrier gas apparatus 10, respectively. An interface b and an interface c of the third three-way selector valve 5 are connected to the first port of the automatic switching six-way valve 11 and an interface b of the second three-way selector valve 4, respectively. An interface a and an interface c of the second three-way selector valve 4 are connected to the sample recovery apparatus 6 and the second port of the automatic switching six-way valve 11, respectively. In the embodiments of the present disclosure, each of the first three-way selector valve 3, the second three-way selector valve 4 and the third three-way selector valve 5 includes the interface a, the interface b, and the interface c, among which the interface a is a common end, and there are two flow directions for a gas path, one is that the interface a communicates with the interface b, and the other is that the interface a communicates the interface c.

The analysis system further includes: a planar tee 9. The carrier gas apparatus 10 is connected to the automatic switching six-way valve 11 by means of the planar tee 9. Specifically, three interfaces of the planar tee 9 are connected to the carrier gas apparatus 10, the interface c of the first three-way selector valve 3, and the fifth port of the automatic switching six-way valve 11. An outlet of the sample storage tank 1 is provided with a first pressure relief valve 2. An outlet of the carrier gas apparatus 10 is provided with a second pressure relief valve 8.

Based on the structure above, the operating principle of the analysis system for hydrocarbon components in ultrapure ammonia is as follows:

Process that a sample does not flow through the electronic refrigeration and concentration apparatus: the sample, after flowing out from the sample storage tank 1, is depressurized by the first pressure relief valve 2, and then flows to the first three-way selector valve 3 (at this time, the interface a communicates with the interface b)→the third three-way selector valve 5 (at this time, the interface a communicates with the interface c)→the second three-way selector valve 4 (at this time, the interface a communicates with the interface b)→the sample recovery apparatus 6.

Process that a carrier gas does not flow through the electronic refrigeration and concentration apparatus: the carrier gas, after flowing out from the carrier gas apparatus 10, is depressurized by the second pressure relief valve 8, flows to the planar tee 9, and then flows from one interface of the planar tee 9 to the first three-way selector valve 3 (at this time, the interface a communicates with the interface c)→the third three-way selector valve 5 (at this time, the interface a communicates with the interface c)→the second three-way selector valve 4 (at this time, the interface a communicates with the interface b)→the sample recovery apparatus 6.

Pipeline purging process: the carrier gas, after flowing out from the carrier gas apparatus 10, is depressurized by the second pressure relief valve 8 and flows to the planar tee 9, and then flows from one interface of the planar tee 9 to the first three-way selector valve 3 (at this time, the interface a communicates with the interface c)→the third three-way selector valve 5 (at this time, the interface a communicates with the interface b)→the first port and the sixth port of the automatic switching six-way valve 11, the electronic refrigeration and concentration apparatus 7, the third port and the second port of the automatic switching six-way valve 11→the second three-way selector valve 4 (at this time, the interface a communicates with the interface c)→and the sample recovery apparatus 6.

Sample collection process: in this process, the automatic switching six-way valve 11 is in the first state, as shown in FIG. 1, the sample, after flowing out from the sample storage tank 1, is depressurized by the first pressure relief valve 2, and then flows to the first three-way selector valve 3 (at this time, the interface a communicates with the interface b)→the third three-way selector valve 5 (at this time, the interface a communicates with the interface b)→the first port of the automatic switching six-way valve 11, the sixth port of the automatic switching six-way valve 11, the electronic refrigeration and concentration apparatus 3, the third port of the automatic switching six-way valve 11, the second port of the automatic switching six-way valve 11→the second three-way selector valve 4 (at this time, the interface a communicates with the interface c)→and the sample recovery apparatus 6.

Analysis and detection process: in this process, the automatic switching six-way valve 11 is in the second state, as shown in FIG. 2, the carrier gas, after flowing out from the carrier gas apparatus 10, is depressurized by the second pressure relief valve 8 and flows to the planar tee 9, and flows from another interface of the planar tee 9 to the fifth port and the sixth port of the automatic switching six-way valve 11, and carries the sample in the electronic refrigeration and concentration apparatus 7 to enter the chromatographic column 12 from the third port and the fourth port of the automatic switching six-way valve 11, and then the sample is separated by the chromatographic column 12 into methane, acetylene, ethylene, ethane, propylene and propane to be detected by the hydrogen flame ionization detector 13.

According to specific embodiments of the present disclosure, the present disclosure has the following technical effects:

The sample is injected through the automatic switching six-way valve 11, and with the cooperation of the electronic refrigeration and concentration apparatus, the content of hydrocarbons (C₁-C₃) is detected through hydrogen flame ionization detection, and the cost of hydrocarbon detection is reduced.

The embodiments in this specification are all described in a progressive manner, each embodiment focuses on a difference from other embodiments, and for same or similar parts in the embodiments, reference may be made to these embodiments. Specific examples are used herein for illustration of the principles and implementation methods of the present disclosure. The description of the embodiments is merely used to help illustrate the method and its core principles of the present disclosure. In addition, a person of ordinary skill in the art can make various modifications in terms of specific embodiments and scope of application in accordance with the teachings of the present disclosure. In conclusion, the content of this specification shall not be construed as a limitation to the present disclosure.

What is claimed is:

1. An analysis system for hydrocarbon components in ultrapure ammonia, comprising a sample storage tank, an electronic refrigeration and concentration apparatus, a carrier gas apparatus, and a detection apparatus;
the electronic refrigeration and concentration apparatus is connected to the sample storage tank, and is also connected to the carrier gas apparatus and the detection apparatus, respectively;
the electronic refrigeration concentration apparatus is used to refrigerate and concentrate a sample to be detected taken out from the sample storage tank;
the carrier gas apparatus is used to carry the sample to be detected after refrigeration and condensation in the electronic refrigeration and concentration apparatus into the detection apparatus;
the detection apparatus is used to detect hydrocarbon components in the sample to be detected after refrigeration and concentration; and
the detection apparatus comprises a chromatographic column, and a hydrogen flame ionization detector.

2. The analysis system for hydrocarbon components in ultrapure ammonia according to claim 1, wherein the analysis system further comprises an automatic switching six-way valve;
the automatic switching six-way valve is connected between the electronic refrigeration and concentration apparatus and the sample storage tank, between the electronic refrigeration and concentration apparatus and the carrier gas apparatus, and between the electronic refrigeration and concentration apparatus and the detection apparatus, specifically as follows:
the sample storage tank is connected to a first port of the automatic switching six-way valve;
the electronic refrigeration and concentration apparatus is connected to a sixth port and a third port of the automatic switching six-way valve, respectively;
the carrier gas apparatus is connected to a fifth port of the automatic switching six-way valve; and
the detection apparatus is connected to a fourth port of the automatic switching six-way valve;
in the process of sample collection, the automatic switching six-way valve is in a first state, the first state is that the first port of the automatic switching six-way valve communicates with the sixth port, the second port communicates with the third port, and the fourth port communicates with the fifth port; and
in the process of analysis and detection, the automatic switching six-way valve is in a second state, the second state is that the first port of the automatic switching six-way valve communicates with the second port, the third port communicates with the fourth port, and the fifth port communicates with the sixth port.

3. The analysis system for hydrocarbon components in ultrapure ammonia according to claim 2, wherein the analysis system further comprises a sample recovery apparatus;
the sample recovery apparatus is connected to the second port of the automatic switching six-way valve.

4. The analysis system for hydrocarbon components in ultrapure ammonia according to claim 3, wherein the analysis system further comprises a first three-way selector valve, a second three-way selector valve, and a third three-way selector valve;
the sample storage tank and the sample recovery apparatus are connected to the automatic switching six-way valve by means of the first three-way selector valve, the second three-way selector valve, and the third three-way selector valve, specifically as follows:
an interface a, an interface b and an interface c of the first three-way selector valve are connected to an interface a of the third three-way selector valve, the sample storage tank and the carrier gas apparatus, respectively;
an interface b and an interface c of the third three-way selector valve are connected to the first port of the automatic switching six-way valve and an interface b of the second three-way selector valve, respectively; and
an interface a and an interface c of the second three-way selector valve are connected to the sample recovery apparatus and the second port of the automatic switching six-way valve, respectively.

5. The analysis system for hydrocarbon components in ultrapure ammonia according to claim 4, wherein the analysis system further comprises: a planar tee;
the carrier gas apparatus is connected to the automatic switching six-way valve by means of the planar tee, specifically as follows:
three interfaces of the planar tee are connected to the carrier gas apparatus, the interface c of the first three-way selector valve and the fifth port of the automatic switching six-way valve, respectively.

6. The analysis system for hydrocarbon components in ultrapure ammonia according to claim 5, wherein an outlet of the sample storage tank is provided with a first pressure relief valve.

7. The analysis system for hydrocarbon components in ultrapure ammonia according to

5. wherein an outlet of the carrier gas apparatus is provided with a second pressure relief valve.
